(19) United States
(12) Patent Application Publication
(10) Pub. No.: US 2026/0232261 A1
(43) Pub. Date: Aug. 13, 2026

(54) Biomarkers of Inflammation in Neurophysiological Systems

(71) Applicant: Massachusetts Institute of Technology, Cambridge, MA (US)

(72) Inventors: Thomas Francis Quatieri, Newtonville, MA (US); Jeffrey Palmer, Westford, MA (US); Tanya Talker, Cambridge, MA (US)

(21) Appl. No.: 19/321,458

(22) Filed: Sep. 8, 2025

Related U.S. Application Data
(63) Continuation of application No. 17/226,442, filed on Apr. 9, 2021, now abandoned.
(60) Provisional application No. 63/007,836, filed on Apr. 9, 2020.

(51) Int. Cl. A61B 5/00; A61B 5/053; A61B 5/08; A61B 7/00; G10L 25/66

(52) U.S. Cl. CPC A61B 5/4803; A61B 5/0075; A61B 5/053; A61B 5/08; A61B 5/7264; A61B 7/003; A61B 7/008; G10L 25/66; A61B 2562/0204; A61B 2562/0219

(57) ABSTRACT

A multi-modal approach is used to detect and track inflammation, and identify location of inflammation, which is revealed in changes in tissue and physiological properties that underlie sub-systems, including respiratory, sinus, laryngeal, articulatory, facial and gastrointestinal components.

### CROSS-REFERENCE TO RELATED APPLICATIONS

This application is a continuation of U.S. application Ser. No. 17/226,442, filed on Apr. 9, 2021, which claims the benefit of U.S. Provisional Application No. 63/007,836, filed on Apr. 9, 2020, which is incorporated herein by reference.

### STATEMENT AS TO FEDERALLY SPONSORED RESEARCH OR DEVELOPMENT

This invention was made with Government support under Contract No. FA8702-15-D-0001 awarded by the U.S. Air Force. The Government has certain rights in the invention.

### BACKGROUND OF THE INVENTION

This invention relates to use of biomarkers for detection of inflammation, and more particularly to use of vocal biomarkers in detection of particular types of respiratory inflammation, for example, as might be found in COVID-19.

Inflammation is a major health issue in civilian and military populations in response to injury, infectious disease, health disorders, and environmental exposure. The inflammation can often occur anywhere in the body, including the respiratory, sinus, musculoskeletal, gastrointestinal, and central/peripheral nervous systems. While inflammation has beneficial protective and recuperative functions, too little inflammation can compromise the immune system and lead to tissue damage with injury, and excessive or chronic inflammation may lead to or be associated with long-term physiological (e.g., muscle) and neurological disease (e.g., autism) and/or other problems in psychological health (e.g., depression).

Although inflammatory responses can occur separately in these bodily systems, more often responses are coupled and modulate each other, sometimes resulting in severe concomitant physiological, neurological, and neurodevelopmental disease. For example, gut inflammation is known to modulate the central nervous system in a way that can initiate or exacerbate Parkinson's disease, autism, or depression. In detecting inflammation or its consequences, manifestations of the condition may not be symptomatic; for example, early stages of inflammation in the gastrointestinal or central nervous system does not always manifest in pain (i.e., due to reduced concentration or absence of pain receptors) and thus may be difficult to detect. Another current, highly relevant example is inflammatory response as a result of respiratory infection associated with COVID-19 which initially can often be asymptomatic in its early stages. Early warning is needed in the form of nonintrusive objective biomarkers that are sensitive (and specific) to subtle variations in neurophysiology due to inflammation in upper and lower respiratory tracts, while also able to track progression of a condition when symptoms are present, and subsequently during treatment and recovery.

### SUMMARY OF THE INVENTION

In one general aspect, a multi-modal approach is used to detect and track inflammation, and identify the location of inflammation, which is revealed in changes in tissue and physiological properties that underlie sub-systems, including respiratory, sinus, laryngeal, articulatory, facial and gastrointestinal components. The approach is also applicable to inflammation of the lower diaphragm, which also underlies vocal subsystems, and may be responsible in part for breathing dysfunction and coughing. In at least some examples, the biomarkers used reflect changes in function and coordination of coupled modalities, measured through, for example, acoustic, optical, ultrasound, and infrared measures of vocal subsystems and underlying physiology. The application can provide early warning and persistent assessment of a variety of inflammatory conditions and their pathological phenotypes.

In some examples, the biomarkers involve physiological and neurocomputational models of the inflammation process and the coupling that occurs across modalities. Given the nonintrusive nature of the sensing, the approach provides a key capability for scalable, longitudinal studies that seek to capture human behavior dynamics in naturalistic environments. As an illustration of the approach, preliminary results distinguishing pre-COVID-19 and post-COVID-19 (pre-symptomatic) conditions, utilize vocal features exploiting change in respiratory function and its relationship with laryngeal and articulatory motion due to post-COVID-19 immune response and associated inflammation. These features could be potentially measured non-intrusively through mobile devices such as smartphones and tablets. Use cases include early warning and monitoring of COVID-19.

In another aspect, in general, an approach to characterizing a potential inflammatory condition in a subject involves acquiring a plurality of concurrent time signals, the time signals characterizing a plurality of physiological or neurological subsystems of the subject. Biomarkers are generated from the acquired time signals (e.g., by a computational procedure from numerically represented time signals). At least some of the biomarkers characterize a time correlation between signals characterizing multiple different subsystems. The biomarkers are then processed (e.g., again with a computational procedure) to yield a characterization of the potential inflammatory condition from the generated biomarkers.

Aspects can include one or more of the following features.

At least some of the time signals representing an acoustic signal produced via the subject's vocal tract.

The characterization of the potential inflammatory condition is provided to a clinician for providing medical services to the subject.

The subsystems comprise two or more subsystems from a group consisting of a respiratory, a sinus, a laryngeal, an articulatory, a facial and a gastrointestinal subsystem.

Acquiring the time signals comprises acquiring signals of two of more types of signals from a group consisting of acoustic, optical, ultrasound, infrared, accelerometer, and impedance or conductance signals.

An advantage of at least some examples of the approach is that acquiring the biomarkers is not intrusive and the biomarkers are simple to measure, exploiting the coupled nature of the relevant neurophysiological systems, while being specific to the underlying inflammation source. One such system comprises the many sub-systems responsible for vocal production, including the laryngeal and articulatory mechanisms during speaking and/or breathing, and is part of a much larger more complex coordinated physiological and neurological network. For example, the coupling of vocal and facial measures with other neurophysiological measures (e.g., respiration, sinus, gut) provides a sensitive indicator of inflammation, both for early warning and long-term, persistent monitoring of inflammation, of great importance to both military and civilian populations.

Yet other advantages of one or more of the described approaches are as follows:

The analysis of sub-components across multiple modalities provides a means to phenotype sources of inflammation that will help identify possible sub-groups of disease: respiratory, congestive, neurological, physiological, etc. As of now, there are no known methods to objectively identify and monitor such sub-groups.

The biomarkers are sensitive in detecting inflammation during the asymptomatic duration of a condition, an important recent example being respiratory change due to COVID-19.

The biomarkers can potentially discriminate between comorbidities (e.g., neuromotor retardation from depression vs infection/inflammation).

Specificity of identification of (and/or ranking of) physiological systems affected (respiratory, sinus, gastrointestinal, nervous, musculoskeletal, etc.) that may differentiate between aches/pains associated with the cold/flu vs neurological disorders, as well as physiological differentiation.

Quantification of reflex arc response to assess functional impact on behavioral abilities/tasks. Quantification of efficacy of therapeutic interventions such as pharmaceuticals (e.g., anti-inflammatorysical therapy, cognitive/behavioral therapy, etc.

In the case of COVID-19 the approaches may address public health response gaps including one or more of the following:
1. Pre-symptomatic & asymptomatic infection screening of at-risk health care workers to prevent COVID-19 transmission
2. Distinguishing more common flu or flu-like symptoms from COVID-19
3. Prioritization of diagnostic test kits and medical services to people at high risk of becoming critically ill during self-isolation
4. Assurance that infection has run its course prior to leaving quarantine and returning to health care work
5. Rapid testing for the masses

Other public health impacts may include one or more of the following:
1. Pre-symptomatic feature tracking: monitor presumptive host's physiological response to the disease to cue self-isolation
2. High specificity of vocal subsystem-based biomarkers will allow important phenotyping across conditions
3. Presumptive host symptom tracking during self-isolation: triage decision support for diagnostic testing and close-contact medical attention
4. Post-quarantine recovery assessment: aid decision support for 2nd COVID diagnostic test and release from quarantine
5. Voice and face measurements on mobile technology is ubiquitous making the approach easily accessible

Another advantage is that the markers that are present for pre-symptomatic subjects (i.e., they have a positive COVID diagnostic test but appear to be asymptomatic), and the disclosed monitoring capability allows for also tracking progression with symptoms present and through recovery when symptom subside but the virus is still present (but subsiding).

The approach is also amenable to tracking severity of COVID-19 through vaccine trial studies.

In some examples, a subject follows a prescribed data collection protocol, which may be designed to elicit different types of activity thereby sampling the systems more predictably. Following such a protocol may provide more consistency between subjects, thereby improving the modeling of the data.

Other features and advantages of the invention are apparent from the following description, and from the claims.

### DESCRIPTION OF DRAWINGS

FIG. 1 is a block diagram.

FIG. 2 is a diagram illustrating functional components and their inter-relations that contribute to signal characteristics.

FIG. 3 is a signal production model illustrating certain subsystems and potential points of coordination.

FIG. 4 are graphs of COVID-19 results.

### DETAILED DESCRIPTION

### 1 Overview

An approach to detection, assessing the severity of and/or determining characteristics of an inflammatory process in a subject involves acquiring time series measurements that represent a number of different physiological/neurological subsystems of the subject. Time correlation characteristics of the measured time series are then used as objective biomarkers that are input to a classification or scoring component, which may be based on machine learning principles. Examples of correlation characteristics include eigenspectra of correlation or covariance matrices formed with time delays of time series from different groups of subsystems.

In some embodiments, the objective biomarkers are within and across vocal and speech sub-systems using measurement of other related modalities to detect and monitor the progression of inflammation under the hypothesis that the inflammatory process measurably affects the functional response of the related nervous and musculoskeletal systems and sensorimotor reflex arc dynamics. These objective, noninvasive biomarkers will reflect response dynamics as well as coordination (correlation) of the temporal dynamics of vocal/speech sub-components within a modality and also of vocal/speech sub-components across modalities.

Measurement modalities may include not only acoustic (speech) and optical (face, motion) approaches but also ultrasonic imaging (which could quantify changes in tissue density and thickness at different depths, potentially due to inflammation) and thermal (facial and body) through infrared imaging, electrophysiology through heart rate, skin conductance, EEG measurements, and vision status through eye tracking and pupillometry measurements, and correlations across these modalities and their sub-components. Use of an acoustic/speech modality is not essential, and only a combination of other modalities can be used. Even if an acoustic/speech modality is not used for a time signal, the other modalities may be acquired during the subject speaking (or breathing/coughing in particular ways), for example, speaking passages from a predetermined protocol, but it should also be recognized that such speaking is not required.

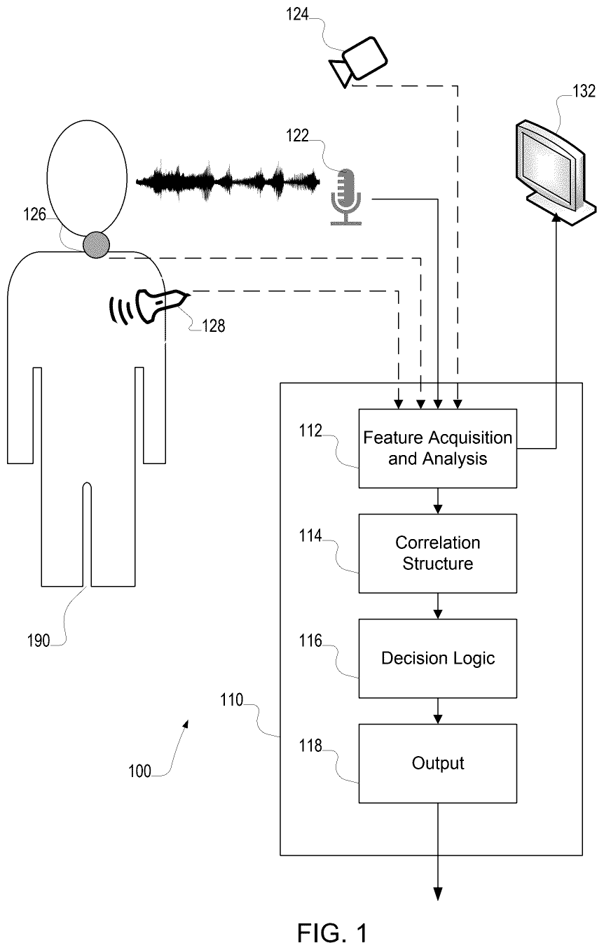

Referring to FIG. 1, an exemplary system 100 for measurement and analysis for signals acquired from a subject 190 makes use of one or more sensors. In general, one of the sensors is a microphone 122, which acquires sounds produced by the subject. Signals (e.g., electrical, digital, etc.) representing the acquired acoustic signal are passed to an analysis system 110. In FIG. 1, the analysis system is schematically illustrated to be in proximity to the subject, but it should be understood that some or all of the system may be remote from the subject. For example, the signals acquired from the subject (or other derived signals) may be transmitted over a communication network to be analyzed. In FIG. 1, a number of alternative sensors are illustrated, without intending that these alternative sensors are required, or to suggest that these are the only other sensors that could be used. For example, a camera 124 may be used to acquire images from which the subject's pupil diameter is determined, or from which other eye movement signals are determined. As another example, an accelerometer or contact microphone 126 may be placed on the subject's throat or other location where acoustic measurements, or other movement measurements, may be made. As yet another example, an ultrasound sensor 128 may acquire signals from which time-varying characteristics of the subject's airways, vocal tract, etc. may be determined.

Continuing to refer to FIG. 1, in general there is an output device, illustrated as a screen 132. The output device is optional, and can be used to prompt the subject to produce various speech or non-speech sounds, or to perform other movements, during which signals are acquired. Other ways of prompting the user can be employed, for example, using printed instructions or audio instructions (e.g., “repeat after me”).

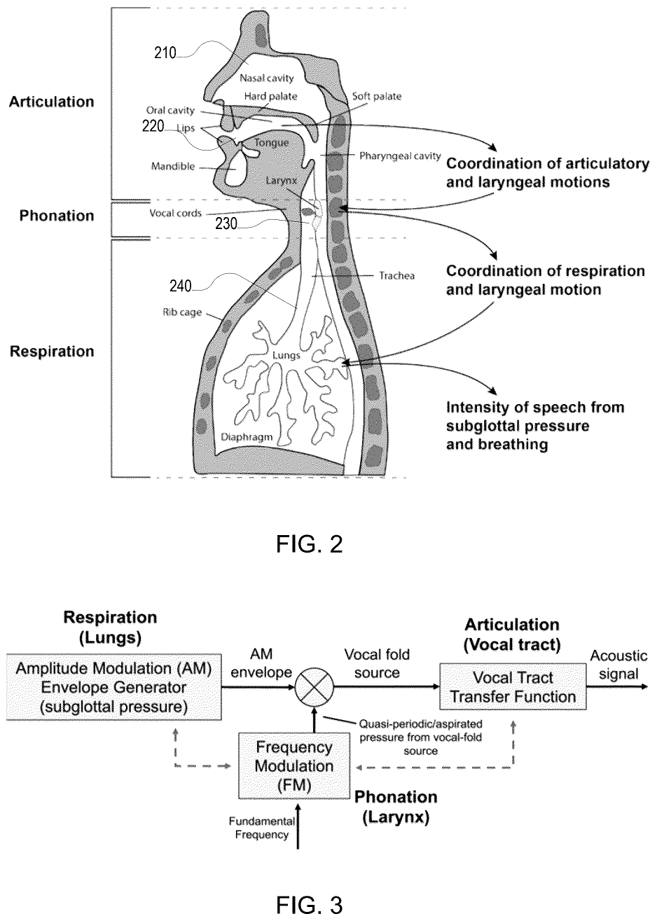

The analysis system 110 in FIG. 2 is shown to have a number of functional components, recognizing that in at least some embodiments, the functions are implemented using a general-purpose computer and these different functional components correspond to different parts of software stored on a machine-readable medium that when executed by a processor performs the functions of those components. A feature acquisition and analysis component 112 receives the signal and preforms any “low level” feature extraction, for example, to extract/compute features introduced above and/or discussed more fully below. A correlation structure component 114 processes the low-level features to determine high-level features, which include correlation structure features of the low-level features. A decision logic component 116 processes the high-level features according to predetermined logic, a statistical decision model, and/or a machine learning element (e.g., artificial neural network, ANN) to determine an output, which may represent a categorical decision (e.g., a condition is present or absent) or a quantitative output (e.g., a score or a probability representing a certainty that a condition is present). For example, a two-class discriminator (e.g., an ANN) that inputs the high-level features may be trained on data for subjects with a condition (e.g., COVID-19) and subjects without that condition. Similarly, a decision logic may be used to predict a change in condition for a subject by providing data acquired at different times to the system.

### 2 Inflammation as Revealed in Speech Production

Physical indicators of inflammation can present themselves in many ways in speech production and related mechanisms. As an example, nasal and lung congestion occurs as a fluid buildup and swelling not only in the nasal and/or lung cavities, but also the oral tract and vocal fold tissues within the larynx. Though obvious in late stages, there is difficulty in early identification of congestive and other ailments, especially during phases while the subject is asymptomatic but still infectious. This is also important in monitoring progression during and after treatment (i.e., prior to release from quarantine). Nasal and lung congestion is one of many examples where inflammation can affect multiple sub-components of a peripheral motor or a physiological system (in this case, the nasal, oral, respiratory, and laryngeal sub-components).

Referring to FIG. 2, to separate out these speech production sub-components, signal processing is applied to both acoustic and non-acoustic microphone time signals. This provides a way to estimate parameters of all four sub-components (nasal 210, oral 220, laryngeal 230, and respiratory 240): Movement of anti-resonances of the nasal cavity and resonances of the oral cavity due to blockage of the nasal cavity, change in the “speaker's resonance” due to fluid buildup in the epilaryngeal cavity above the vocal folds, and modulation of respiration and pitch due to lung fluid and/or neuromuscular tension due to inflammation in regions surrounding the vocal folds. Changes in airflow through the glottis (space between the folds) can also be measured through inverse filtering of the acoustic signal or more directly through non-acoustic measurements (EEG) and ultrasonic imaging (to obtain tissue density and thickness around the glottis and also in the nasal and oral cavities). Changes of features both within and across these sub-components can also be obtained. With the underlying inflammation condition, fluid buildup and swelling can also occur in the face, as well as other body parts, effecting fine and gross motor movement and physiological response such as heart rate and skin conductance.

### 3 Feature Extraction

In an exemplary embodiment, the system makes use of a number of low-level features that are measured based on acoustic, or other, signals acquired from the subject. These features are selected based on the physiologically motivated speech production model in FIG. 3 where the airflow from the lungs during the exhalation phase of speech production passes through the bronchial tubes through the trachea and into the larynx. The ‘intensity’ of the airflow (velocity), referred to herein as the respiratory intensity, governs time-varying loudness, and is coupled (coordinated) with phonation, i.e., the vibration of the vocal folds (fundamental frequency or ‘pitch’), stability of phonation, and aspiration at the folds. These characteristics are a function of laryngeal muscles and tissue, modulated by the respiratory intensity. Finally, according to this model, the vocal fold source signal is modulated by, and coordinated with, the vocal tract movement during articulation.

Low-level univariate features characterize basic properties of the three vocal subsystem components. The speech envelope is used as a proxy for respiratory intensity and is estimated using an iterative time-domain signal envelope estimation algorithm, providing a smooth contour derived from amplitude peaks. At the laryngeal level, an estimate of the fundamental frequency (pitch) is determined using an autocorrelation approach and cepstral peak prominence (CPP) is computed, representing stability of vocal fold vibration. CPP is based on the ratio of the pitch-related cepstral peak relative to aspiration noise level. CPP may be computed as the difference in dB between the magnitude of the highest peak and the noise floor in the power cepstrum (the result of taking the inverse Fourier transform of the logarithm of the estimated spectrum of a signal). As a measure of vocal fold stability, CPP has the potential to reflect change in the coupling of subglottal respiratory and laryngeal subsystems. Finally, as a proxy for articulation, a primary feature set comprises the vocal tract resonances (referred to as formant frequencies) estimated by a Kalman filter technique, smoothly tracking the first three formant frequencies while also smoothly coasting through nonspeech regions. Implementations may make use of the open source software package KARMA to compute the first three formant frequencies (F1, F2, F3). These low-level features are computed only during speaking using a speech activity detector. Other features that may be used include the harmonics-to-noise (HNR) ratio, which is the ratio, in decibels (dB), of the power of the harmonic (periodic) signal from vocal-fold vibration and the power of the speech noise signal at the vocal folds created by turbulence as air rushes past the vocal folds from the lungs. This measure is thought to reflect breathiness in a voice. Another measure is termed “creak,” corresponding to what is often referred to as creaky voice, which reflects large irregularity in pitch periods (often with low average pitch) and high peakiness of airflow pulses that excite the vocal tract. The value here is given as a creak probability.

Another feature that may be used is the Glottal Open Quotient (GOQ), which is the ratio of the time duration over which the folds are open relative to the full glottal cycle. A larger open quotient often results in more turbulent airflow at the folds. Implementations of various of the feature extraction procedures may use open source software packages, for example, Praat and VoiceBox.

A number of high-level features are derived from low-level features, and are designed to capture coordination of the temporal dynamics of speech production subsystems at different time scales. The high-level features include computation of multivariate auto-and cross-correlations (or covariances) within and across the underlying speech subsystems. We refer to these features as a “correlation structure.” Specifically, time-delay embedding is used to expand the dimensionality of the feature time series, resulting in a correlation matrix with embedded auto-and cross-correlation patterns that represents coupling strengths across feature channels at multiple time delays. The eigenspectrum of the correlation matrix quantifies and summarizes the frequency properties of the set of feature trajectories.

Higher complexity of coordination across multiple channels is reflected in a more uniform distribution of eigenvalues, and more independent ‘modes’ of the underlying system components, while lower complexity is reflected in a larger proportion of the overall signal variability being concentrated in a small number of eigenvalues. In the latter case, the eigenspectra concentration typically manifests with high-rank eigenvalues being lower in amplitude and thus reflecting more dependent or ‘coupled’system components.

For each speech segment, correlation matrices are calculated from various combinations of feature trajectories from the different speech subsystems. Each matrix contains the correlation coefficients between the time-series at specific time delays to create the embedding space. Each matrix is computed at a specific delay scale (e.g., 10, 30, 70, or 150 ms) with 15 time-delays used per scale. The delay scales allow for characterization of coupling of signals at different time scales. In a preferred embodiment, eigenspectra are computed with a 10 ms delay scale and thus a relatively fine time resolution. Each matrix comparing n signals has a dimension of (n*15×n*15). For all correlations, an automatic masking technique was used to include only voiced speech segments.

In assessing motor coordination, the eigenvalues of the correlation matrix across individual segments that made up the session are averaged to obtain the mean eigenspectra for the entire data collection for a subject (e.g., the extent of an interview with the subject). Under various conditions, different degrees of dynamical complexity both within (e.g., respiration intensity, formant frequencies) and across features (e.g., respiration vs fundamental frequency), are generally found in these features dependent on the condition, as reflected in eigenvalue distributions.

### 4 COVID-19 Example Case Study

Using the present multi-modal approach, an objective is to detect and monitor COVID-19 with vocal features to characterize coughing and breathing (with or without speech), location of inflammation (e.g., lung versus sinus for specificity), and changes in motor function of speech subsystems due to inflammation. Breathing is characterized not only by the nature and intensity of respiration but also by words per breath group/intensity while speaking (vs simple breathing), respiration in speaking hypothesized to provide a more sensitive measure of breathing abnormalities. Inflammation location may be determined through anti-resonances in vocal tract due to upper (nasal) vs lower (multi-point lung) regions using our multi-modal approach. The effect of inflammation on the complexity of motor coordination of different speech subsystems will be determined. FIG. 2 depicts these subsystems and the coordination across subsystems that are hypothesized to be affected.

In one approach to data acquisition, the subject is instructed to produce various sounds, and during the production of those sounds, the various sensor signals (e.g., the acoustic signal acquired by a microphone, possibly augmented by other sensor signals) are acquired. Various instructions may be provided. As one example of a data collection protocol, the subject receives the following instructions.
1a) Read at your own natural pace the following passage (“The Caterpillar” from Rupal Patel et al. (2013):“
   “Do you like amusement parks? I sure do. I went twice last spring. My BEST moment was riding the Caterpillar, which is a huge rollercoaster. I saw how high the Caterpillar rose into the bright blue sky. I waited in line for thirty minutes. The man measured my height to see if I was tall enough. I gave the man my coins, and jumped on the cart. It went SO high I could see the parking lot. Boy was I SCARED! As quickly as it started, the Caterpillar came to a stop. Unfortunately, it was time to pack the car and drive home.”
1b) Now read the above passage as fast as you can,
2) Read each of the following sentences in 3 different ways:
   Natural
   Inhale and in one exhalation, recite both sentences in one breath
   Exhale and then at end of the exhalation recite both sentences
The sentences (the “K-mart sentences”, attributed to Louis Goldstein and Catherine Browman, contain all phonemes of English (minus affricates)):
   “The girl was thirsty and drank some juice, followed by a coke.”
   “Your good pants look great, however, your ripped pants look like a cheap version of a K-mart special. Is that an oil stain on them?”
3) Read the following sentences (sentences with high frequency of nasals):
   “Let me sing by the moon with novel phonemes.”
   “There is nothing in my spoon to use as a sling.”
4) Say and hold each of the following vowels for 10 seconds:
   /o/, as in “hot”
   /E/, as in “feet”
   /OO/, as in “moon”
   /a/, as in “sack”
5) Say and hold each nasal for 10 seconds
   /n/, as in “spoon”
   /m/, as in “mop”
   /ng/, as in “sing”
6) Repeat the following as many times as possible in one breath:
   “pa-ta-ka”
7) Answer a few questions
   “Describe your favorite food. What ingredients are in it? Why do you like it? What memories do you associate with it?”
8) Deep breaths
   Inhale for 4 seconds, then exhale for 4 seconds
9) Force a cough
   Two coughs each breathe (repeat twice)
10) Hold your nose pinched and recite:
   “The girl was thirsty and drank some juice, followed by a coke.”

These instructions are only an example, and other data acquisition protocols may be used. For example, speech produced during a single passage or recording, or during a conversation may be used.

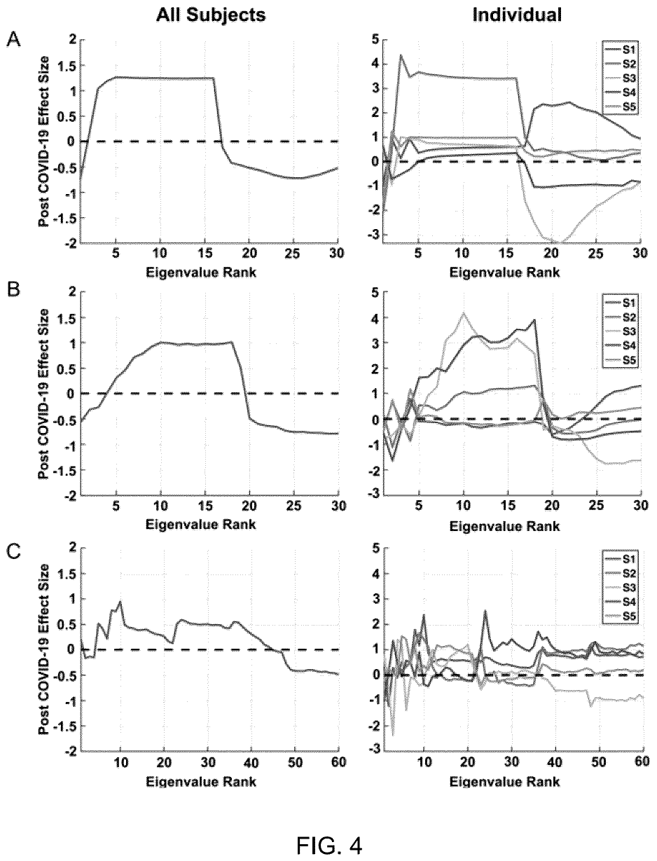

An example of results in the case of COVID-19 are shown in FIG. 4. Generally, these graphs show coordination of respiration with laryngeal characteristics, as well as coordination of laryngeal characteristics with articulation. Group and subject-dependent effect sizes are shown in left and right columns, respectively. (A): respiratory intensity (speech envelope) and pitch (vocal fold fundamental frequency)—30 eigenvalues obtained from 2 features×15 correlation samples; (B): respiratory intensity and stability of vocal fold periodicity (CPP)—30 eigenvalues obtained from 2 features×15 correlation samples; (C): pitch and articulation (first 3 formant center frequencies)-60 eigenvalues obtained from 4 features×15 correlation samples. Effect sizes greater in magnitude than 0.37 in the comparison across all subjects have corresponding p<0.05. The graphs in FIG. 4 are derived from pre-and post-COVID-19 recordings of five subjects (S1 to S5). The right-hand panels show Cohen's d effect sizes for each of the subjects, and the left-hand panels show a combination of results for the subjects. Each row of panels is associated with one of three measures of coordination: respiratory intensity (speech envelope) and pitch (vocal fold fundamental frequency), respiratory intensity and stability of vocal fold periodicity (CPP), and pitch and articulation (first 3 formant center frequencies). Effect size patterns for the two cases involving respiration show similar high-to-low trends across many of the subjects, with high-rank eigenvalues tending toward relatively lower energy for the post-COVID-19 cases. Effect sizes for the combined subjects indicate a similar but more distinct group-level morphology in these cases. On the other hand, effect sizes for coordination of pitch (fundamental frequency) and articulation (formant center frequencies) is more variable across subjects, but the combined counterpart shows a high-to-low trend, albeit weaker than those involving respiration. Although a strict interpretation is not possible due to the small cohort, at a group level, the morphology of effect sizes in FIG. 4 indicates a reduction in the complexity of coordinated subsystem movement, in the sense of less independence of coordinated respiratory and laryngeal motion and likewise, but to a lesser extent, for laryngeal and articulatory motion.

Although the group-level eigenspectra-based effect size trends indicate a reduced complexity in coordination, clearly a larger cohort is warranted as well as addressing a number of confounders, including subject-and recording-dependences, in any validation procedure. For example, across all variables, inter-subject analysis shows for one subject a distinctly different trend of larger high-ranking eigenvalues that indicates a more complex but more erratic (or variable) coordination. Regarding signal quality, due to the nature of the online video sources, there is a variety of inter-and intra-subject recording variability, the most perceptually notable effect being reverberation, possibly modifying the true effect sizes, over-or under-estimating their importance. An example given in the Supplementary Material, isolating two of the subjects with more consistent, least reverberant environments, enhances the combined effect sizes relative to the N=5 case.

### 5 Phenotyping

Novel markers derived from such multi-modal sub-systems (acoustics being one example) will provide persistent assessment of a variety of inflammatory diseases but perhaps more importantly the multi-modal nature of high-temporally and spectrally resolved sub-component measurements will provide a means to discriminate location of the inflammation and phenotype the disease. Refinement of these markers will involve physiological and neural computational modeling of the various sub-system components and their modification due to the inflammatory process and the coupling that occurs across sub-systems. Features derived from these models as well as their associated behavioral measures will provide a means to further phenotype to identify which bodily system and which system sub-components are inflamed (e.g., in speech production: nasal, oral, respiratory, or laryngeal sub-components or in facial expression, eye, nose, month, cheeks, jaw), as well as the degree and location of inflammation within a sub-component (e.g., inflammation of vocal cords, sub-glottal tracheal or super-glottal pharyngeal region, or the location of blockage within the nasal, lung, and tracheal passage). The approach will ultimately provide a key capability for longitudinal studies that seek to capture human behavior dynamics in naturalistic environments during onset and progression.

Implementations of the approaches described above may make use of hardware, software, or a combination of hardware and software. Hardware may include data or signal processing circuitry, for example, in the form of Application Specific Integrated Circuits (ASICs) or Field Programmable Gate Arrays (FPGAs). Software may include instructions stored on non-transitory machine-readable media that are executed by a processor. In some examples, the processor is a general purpose computer processor, for example, embedded in a personal computation device, such as a smartphone or mobile computer/tablet. In some examples, the signals are acquired at one device (e.g., a smartphone), while the processing of the signals is performed on a computation system remote from the signal-acquiring device. In some implementations, the processing of the signals is performed in real time, while in other implementations, the signals are recorded, and processed at a later time. In some implementations, rather than providing results to a clinician, other automated and/or machine implemented methods can be used, for example, to triage subjects, route them in a health care facility, or route a communication session (e.g., telephone call) to an appropriate destination. A number of embodiments of the invention have been described. Nevertheless, it is to be understood that the foregoing description is intended to illustrate and not to limit the scope of the invention, which is defined by the scope of the following claims. Accordingly, other embodiments are also within the scope of the following claims. For example, various modifications may be made without departing from the scope of the invention. Additionally, some of the steps described above may be order independent, and thus can be performed in an order different from that described.

What is claimed is:

1. A method for characterizing a potential inflammatory condition in a subject, the method comprising:
acquiring a plurality of concurrent time signals, the time signals characterizing a plurality of physiological or neurological subsystems of the subject;
generating biomarkers from the acquired time signals, at least some of the biomarkers characterizing a time correlation between signals characterizing multiple different subsystems; and
processing the biomarkers to yield a characterization of the potential inflammatory condition from the generated biomarkers.

2. The method claim 1, wherein at least some of the time signals representing an acoustic signal produced via the subject's vocal tract.

3. The method of claim 1, further comprising providing the characterization of the potential inflammatory condition to a clinician for providing medical services to the subject.

4. The method of claim 1 wherein the subsystems comprise two or more subsystems from a group consisting of a respiratory, a sinus, a laryngeal, an articulatory, a facial and a gastrointestinal subsystem.

5. The method of claim 1 wherein acquiring the time signals comprises acquiring signals of two of more types of signal from a group consisting of acoustic, optical, ultrasound, infrared, accelerometer, and impedance or conductance signals.

6. The method of claim 1 wherein the time signals include an acoustic time signal acquired using a microphone.

7. The method of claim 6, wherein the acoustic time signal comprises a signal contacting speech produced by the subject.

8. The method of claim 1 where the time signals comprise a bio-impedance or conductance signal acquired between locations on the subject's body.

9. The method of claim 8, wherein the impedance or conductance signal is acquired between locations on the subject's torso.

10. The method of claim 8, wherein the impedance or conductance signal is acquired between locations proximal to the subject's larynx.

11. The method of claim 1, wherein generating the biomarkers comprises computing a time delay correlation or a time delay covariance matrix from the time signals.

12. The method of claim 11, wherein generating the biomarkers further comprises computing an eigenspectrum of the correlation or covariance matrix.

13. The method of claim 1, wherein processing the biomarkers includes applying a machine-implemented classification or scoring approach to the biomarkers.

14. The method of claim 13, wherein applying the machine implemented approach comprises using a machine learning approach to yield the characterization of the potential inflammatory condition.

15. The method of claim 14, wherein using the machine learning approach includes determining parameters of the machine learning approach from data collected from a plurality of training subjects with known characteristics of inflammatory condition.

16. The method of claim 1 wherein the inflammatory condition comprises a coronavirus-related condition.

17. The method of claim 16, wherein the coronavirus-related condition comprises COVID-19.

18. The method of claim 1, wherein the characterization of the potential inflammatory condition comprises at least one of a presence or absence, a degree, or a physical location, of the inflammatory condition.

19. A system configured to perform all the steps of claim 1.

20. The system of claim 19, comprising at least one microphone for acquiring at least one of the time signals, and a processor and a storage for instructions that when executed by the processor cause the processor to perform the steps of generating and processing the biomarkers.
